(19) United States
(12) Patent Application Publication
(10) Pub. No.: US 2007/0116680 A1
(43) Pub. Date: May 24, 2007

(54) Stem cells within gel microenvironments

(75) Inventors: Jan P. Stegemann, (US); Evan J. Lord, (US)

Correspondence Address: Dr. Raj Bawa;Bawa Biotechnology Consulting, LLC, 21005 Starflower Way, Ashburn, VA, 20147, US

(73) Assignee: Rensselaer Polytechnic Institute

(21) Appl. No.: 11/521,315

(22) Filed: Sep. 15, 2006

Related U.S. Application Data
(60) Provisional application No. 60/738,172, filed on Nov. 18, 2005.

(51) Int. Cl. A61K 35/12; C12N 5/08; C12N 5/06

(52) U.S. Cl. 424/93.7; 435/325; 435/366

(57) ABSTRACT

This invention provides a system to embed stem cells within three-dimensional (3D) hydrogel microenvironments consisting of naturally derived proteins, proteoglycans and/or polysaccharides. Pure matrices or combinations of materials can be used. The method involves suspending stem cells in solutions of the matrix components of interest, emulsifying these solutions in a hydrophobic phase, triggering gelation of the matrix components by changing the environmental conditions, and collection of the resulting hydrogel beads. The unique bead format of this invention has the advantage of allowing the use of small amounts of rare matrix proteins. Bead preparations can be concentrated into a paste for use as a cell delivery vehicle to damaged tissues, either directly after encapsulation or after a period of culture to promote stem cell differentiation. Defined 3D microenvironments can guide stem cell differentiation, and the resulting beads can be used directly as a cell delivery vehicle in various tissue repair applications.

### CROSS-REFERENCE TO RELATED APPLICATION

This application claims benefit under 35 U.S.C. § 119(e) of U.S. provisional patent application No. 60/738,172, filed Nov. 11, 2005, the disclosure of which is herein incorporated in its entirety by reference.

### TECHNICAL FIELD

The present invention relates generally to the field of cell biology of stem cells and their differentiation, delivery and use in regenerative medicine. More specifically, it describes methods of embedding stem cells within three-dimensional (3D) microenvironments, thereby guiding their differentiation to a desired phenotype.

### BACKGROUND OF THE INVENTION

Stem cells are progenitor cells that have the ability to divide to form additional stem cells, as well as to differentiate into tissue-specific cells. The ability to self renew and differentiate into numerous cell types makes the use of stem cells to repair or replace damaged tissues a promising approach to many clinical problems. However, the chief factor limiting stem cell-based therapies is the inability to control the induction and maintenance of cell differentiation. The invention pertains to a method of embedding living stem cells in controllable hydrogel microenvironments with one or more of the following purposes: (i) maintaining their undifferentiated progenitor state; (ii) promoting their proliferation; (iii) directing their differentiation towards a desired tissue type.

It is well known that cell function is controlled by the entirety of the surrounding three-dimensional (3D) cellular environment. Most tissue cells are surrounded on all sides by a complex set of extracellular matrix (ECM) proteins that are critical in guiding cell function. Cells bind to the ECM via specific integrin receptors, and this binding serves as a chemical cue that can directly affect cell function (Hynes, 2002). In addition, the ECM acts as a modulator of biochemical and mechanical stimuli that are present in tissues. For example, ECM proteins can sequester and release growth factors (Lee and Juliano, 2004), control the rate of nutrient supply, as well as control cell shape and transmit mechanical signals to the cell surface (Katsumi et al., 2004). The mechanical compliance of the ECM that surrounds cells is also an important factor in controlling cell function (Georges et al., 2005). The invention creates a defined microenvironment around the embedded stem cells, such that their function and differentiation can be controlled as in a tissue.

There are two main types of stem cells that are of interest for therapeutic uses are: embryonic stem cells, and adult stem cells. Embryonic stem cells (ESC) are obtained from the undifferentiated inner mass of an early stage embryo (blastocyst). They are considered to have the highest degree of differentiation potential, with the ability to differentiate into any cell type in the body. Adult stem cells have been found in a variety of tissues, and a common source of adult stem cells is the bone marrow. Aspirates of human bone marrow contain both hematopoietic cells as well as stromal cells, which can be separated out by their ability to adhere to culture substrates (Digirolamo et al., 1999). A subpopulation of these stromal cells from adults has been shown to propagate while retaining the ability to express phenotypic characteristics of multiple cell lineages (Barry and Murphy, 2004; Datta et al., 2005; Pittenger et al., 1999). These cells are often referred to as marrow stromal cells or human mesenchymal stem cells (hMSC), and have been shown to differentiate into osteoblasts, chondroblasts, adipocytes, endothelial cells, neurons, smooth muscle cells, skeletal myoblasts, and cardiac myocytes (Pittenger and Martin, 2004).

Bone marrow can be easily harvested from the superior iliac crest of the pelvis using a minimally invasive aspiration technique, and MSC therefore are an attractive autogenous cell source for many clinical applications. However, many of the current methods used to induce desired differentiation of hMSC in vitro involve treatments that are difficult to implement for use as in vivo therapies. For example, in the case of bone repair, hMSC cultured in the presence of ascorbic acid-2-phosphate, b-glycerophosphate, and the synthetic glucocorticoid dexamethasone adopt an osteoblastic phenotype, and secrete and organize an extracellular matrix (ECM) where calcium phosphate is deposited as hydroxapatite crystals. While these media supplements clearly induce osteoblast differentiation under some conditions, they may suppress bone growth in vivo (Ng et al., 2002), which may limit their usefulness for repairing bone in situ. In addition, response to these agents is biphasic, concentration-dependent, and varies with the length of exposure (Aubin, 2001). The result is a somewhat heterogeneous population of cells with mixed differentiation potential (Candeliere et al., 2001). Hence, there is a clear need in the art to better control the differentiation of hMSCs in culture, for bone and other tissue repair applications.

Continuing with the example of bone tissue, it has been shown in vitro that bone marrow-derived stem cells undergo osteogenic differentiation when cultured on a collagen Type I matrix, and that this requires interaction with the collagen binding integrin receptor alpha2beta1 (Mizuno and Kuboki, 2001; Xiao et al., 1998). Other ECM proteins and their integrin receptors contribute to the osteogenic differentiation of bone marrow stromal stem cells as well (Franceschi, 1999; Gronthos et al., 2001). In particular, it has been established that plating hMSCs on collagen Type I, vitronectin, or laminin is sufficient to induce osteogenic differentiation in the absence of any soluble stimuli (Klees et al., 2005; Salasznyk et al., 2004). The recognition that ECM can be a potent regulator of hMSC phenotype has led to efforts to control hMSC function and phenotype by providing appropriate substrates, in both two-dimensional (2D) and 3D culture approaches. Encapsulation of hMSCs in 3D hydrogel materials has been investigated using several systems. Synthetic polymer systems have been created for culture of hMSCs in orthopaedic applications (Temenoff et al., 2004; Wang et al., 2005). In addition, naturally-derived ECM proteins such as collagen Type I and fibrin have been investigated for the culture and controlled differentiation of hMSCs (Awad et al., 2004; Bensaid et al., 2003). The invention is distinct from these previous approaches in that it embeds stem cells in discrete 3D microenvironments that contain controlled hydrogel compositions, composed of naturally-derived proteins, proteoglycans and/or polysaccharides. The ability to include full proteins (as opposed to peptide fragments) in these environments is a key to directing cell function and phenotype. These microenvironments can further be concentrated into a paste for use as a cell delivery vehicle.

### BRIEF DESCRIPTIONS OF THE DRAWINGS

FIG. 1: Schematic showing how directed differentiation of stem cells can be used to create tissue-specific cells for therapeutic applications.

FIG. 2: Schematic diagram of mixer, impeller and reservoir

FIG. 3: Schematic diagram of bead production process

FIG. 4: Size distributions of pure agarose beads produced by varying the system parameters of stirring speed (SS) and blade separation (BS).

FIG. 5: Size distribution of collagen-agarose bead populations made with varying collagen concentrations. A) 0%, B) 5%, C) 12%, D) 25%.

FIG. 6: Light micrographs (LM) and fluorescence images (F) of collagen-agarose beads with collagen concentrations of (a) 0% (b) 5% (c) 12%, (d) 25% AND (E) 40% (wt/wt). The textured appearance of collagen-gelatin beads indicates the incorporation of collagen.

FIG. 7: Morphology of hMSC in collagen-agarose beads at day 8 (0%, 25%, 40% collagen) FIG. 8: Images of various bead formulations: A) gelatin beads, B) collagen-gelatin beads, C) collagen-fibronectin-gelatin beads D) collagen beads, E) chitosan beads, F) collagen-chitosan beads.

FIG. 9: schematic of how gel bead microenvironments can be used for bone repair (generalizable to other tissues).

FIG. 10: Images of gelatin bead paste being extruded from a syringe (Panel A). Preparation is dyed blue for contrast. Note that bead preparation holds its shape well. Panel B shows beads from paste after being resuspended in saline by vigorous agitation. Beads retain their shape.

### DETAILED DESCRIPTION OF THE INVENTION

In this specification, although the preferred embodiments have been described in detail, it should be understood that various changes, substitutions and alterations may be made therein without departing from the spirit and scope of the invention. Therefore, the specification is to be regarded in an illustrative rather than a restrictive sense. In other words, the described embodiments of the invention and the disclosed examples are given for the purpose of illustration rather than limitation of the invention as set forth in the appended claims.

Unless otherwise defined herein, scientific and technical terms used in connection with the present invention shall have the meanings that are commonly understood by those of ordinary skill in the art. For instance, in the case of the naming of proteins (or any other matrix components/compounds), reference to a general protein name is meant to encompass all the subtypes of the protein. In this regard, the term “collagen” refers and applies to all twenty eight known subtypes of collagen known in the art as well as other subtypes should they be discovered. Furthermore, unless otherwise required by context, singular terms shall include pluralities and plural terms shall include the singular. Generally, nomenclatures used in connection with, and standard techniques described herein are those well known and commonly used in the art. Furthermore, unless stated to the contrary, any use of the words such as “including,” “containing,” “comprising,” “having” and the like, means “including without limitation” and shall not be construed to limit any general statement that it follows to the specific or similar items or matters immediately following it. It should also be understood that the various embodiments of the present invention are not mutually exclusive, but may be implemented in various combinations.

Definitions

To facilitate understanding of the present invention, the following terms and phrases are defined herein and used throughout the specification.

The phrase “naturally derived polymer” refers to chemical substances or compounds that have been extracted, obtained or derived from one or more living organism. These substances or compounds are useful in preparing cosmetic or pharmaceutical compositions that are generally non-toxic, and are also used in regenerative medicine applications.

The term “growth factor” refers to a naturally occurring protein capable of regulating a variety of cellular processes. Growth factors can regulate such processes as cellular proliferation, migration, differentiation and survival. For example, bone morphogenetic proteins (BMP) stimulate bone cell differentiation, while vascular endothelial growth factors (VEGF) stimulate new blood vessel formation. Other growth factors of particular interest in this invention include fibroblast growth factor (FGF), epidermal growth factor (EGF), platelet-derived growth factor (PDGF), transforming growth factor (TGF), neural growth factor (NGF), hepatocyte growth factor (HGF), insulin-like growth factor (IGF), interleukin (IL), interferon (INF), tumor necrosis factor (TNF) and colony stimulating factor (CSF).

The term “excipient” means a component or ingredient of a cosmetic or pharmaceutical composition or product that is not the active ingredient contained therein. The excipients that are useful in preparing a cosmetic or pharmaceutical composition are preferably generally safe, non-toxic and neither biologically nor otherwise undesirable, and are acceptable for veterinary use as well as human pharmaceutical use.

As used in this disclosure, the term “stem cell” can refer to either a pluripotent stem cell or a committed precursor cell. Minimally, a stem cell has the ability to proliferate and form cells of more than one different phenotype, and is also capable of self renewal—either as part of the same culture, or when cultured under different conditions. Examples of stem cells are embryonic stem cells, cord blood stem cells and adult mesenchymal stem cells. The phrases “umbilical cord blood” or “cord blood” refer to blood that is obtained from a neonate or a fetus. It preferably refers to blood which is obtained from the umbilical cord or placenta of newborns, often obtained by direct drainage from the umbilical vein.

For the purposes of this description, “differentiated” and “undifferentiated” are relative terms depending on the context in which they are employed. Specifically, in reference to a particular type of self-renewing stem cell, the term “undifferentiated” refers back to the same self-renewing stem cell, whereas the term “differentiated” refers to one or more of the relatively mature phenotypes the stem cell can generate—as observed or detected by morphological criteria, antigenic markers, gene transcripts produced, etc.

The term “hydrogel” refers to a broad class of polymers which are swollen extensively when in contact with water but which do not dissolve in water. Often, hydrogels are synthesized via polymerizing hydrophilic monomers in an aqueous solution under conditions where the polymer becomes cross linked so that a three-dimensional polymer meshwork is formed. Hydrogels may have a net positive or negative charge, or may be neutral.

As used in this description, the terms “encapsulated” and “embedded” refer to the state of being entrapped and/or surrounded by a matrix. In the case of stem cells, encapsulated or embedded cells may be entirely or partially surrounded by matrix material; they may be able to move through, proliferate in and remodel the matrix.

As used in this disclosure, the term “crosslinked” refers to an attachment of two chains of polymer molecules by bridges, composed of either an element, a group, or a compound, that join certain atoms of the chains by primary chemical bonds. Based on this description, the phrase “crosslinked hydrogel system” refers to a three-dimensional network of crosslinked hydrophilic polymers in the form of a gel that is composed of water. The “crosslinked hydrogel system” described herein is preferably in the form of gels, but not limited thereto.

Description

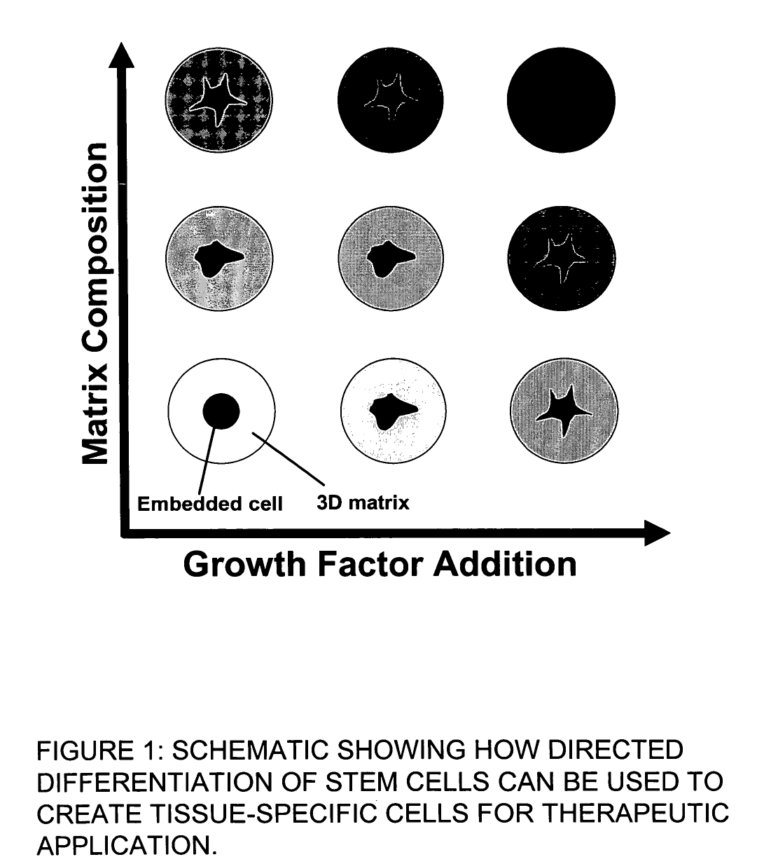

As discussed in detail earlier, reliable control over the process of cell differentiation is a major challenge in moving stem cell-based therapies forward. The composition of the ECM plays an important role in modulating this differentiation. Therefore, the ability to vary the composition of 3D microenvironment allows control of the insoluble biochemical cues to which the cells are exposed via cell-matrix interactions. Controlling these signals will ultimately drive the differentiation of stem cells towards desired cell types. This general concept may be applied to a variety of stem cell types (including embryonic, cord blood and mesenchymal stem cells) because the differentiation and functions of these stem cell types is controlled by the extracellular environment. FIG. 1 shows schematically how control of the 3D microenvironment surrounding an embedded stem cell might be used to promote desired tissue formation. Controlled alterations of the matrix composition (vertical axis) or growth factor level/type (horizontal axis) causes a change in cell phenotype, represented here by a change in cell spreading. The associated change in cell function leads to formation of the desired tissue type (e.g. bone, cartilage, muscle, nerve, etc.), as represented by a darkening matrix.

Accordingly, this invention discloses a unique system to encapsulate adult hMSCs within spherical 3D microenvironments resulting in naturally derived protein-based, stem cell-containing hydrogel beads. These hydrogel beads can potentially be used as defined, 3D microenvironments for the directed differentiation of stem cells. The protein(s) can be selected from, but not limited to, collagen (including Types I, II, III and IV), fibrinogen, fibrin, fibronectin, gelatin, laminin, and vitronectin. In addition, the gel microenvironments can contain proteoglycan components (including hyaluronan and heparan sulfate), as well as polysaccharide components (including agar, agarose, alginate, chitosan and various combinations thereof). These protein-based beads are produced by creating a suspension of stem cells in a liquid solution of the matrix of interest and then emulsifying that suspension in a hydrophobic fluid phase. Subsequent triggered gelation of the matrix causes hydrogel beads to form, which are collected for use. Both the bead size and protein concentrations can be easily controlled by the disclosed methods. High cell viability post-encapsulation for all bead formulations is consistently observed. Therefore, the applicants' system of producing 3D microenvironments of defined matrix composition offers a novel way to control cell-matrix interactions and thereby guide stem cell differentiation. Furthermore, the unique bead format of the instant invention allows the use of small amounts of matrix proteins, and such beads can potentially be used as a cell delivery vehicle in various tissue repair applications.

### EXAMPLE 1

### Collagen-Agarose Beads

In one embodiment of the present invention, hMSC were directly embedded into the matrix of 3D microbeads consisting of varying amounts of agarose and collagen Type I. The inventors examined factors that influence the bead production process, as well as the effects of varying matrix composition on hMSC viability and morphology. Collagen Type I was used because of its established involvement in influencing the phenotype of hMSCs, in particular towards the osteoblastic lineage. Agarose was used as an inert filler material to provide structural integrity to the beads and facilitate bead harvesting. By varying the ratio of agarose to collagen, the effect of ECM on hMSC function could be examined.

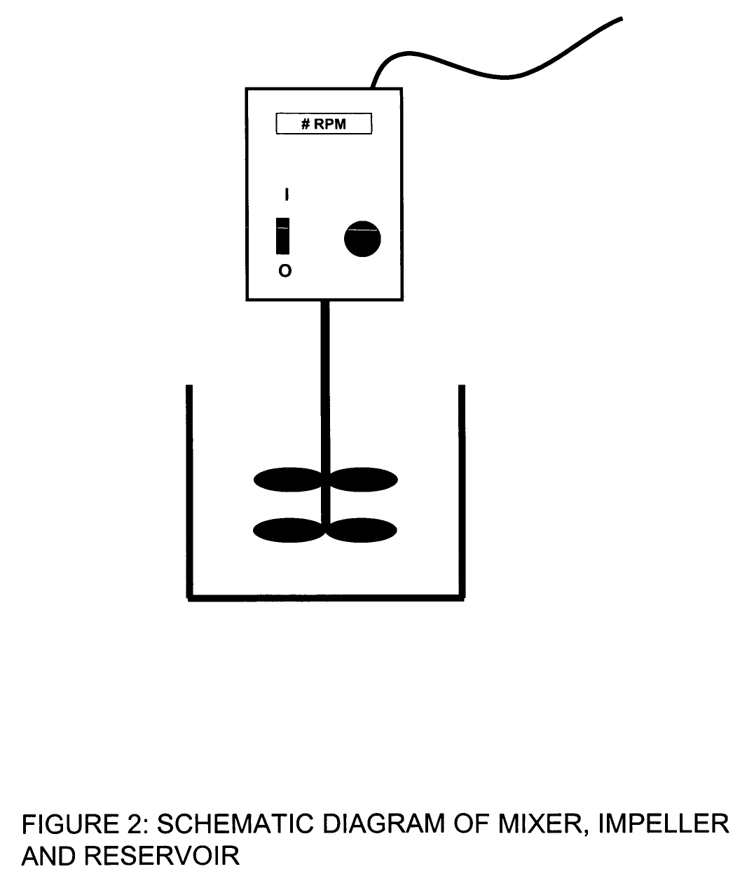

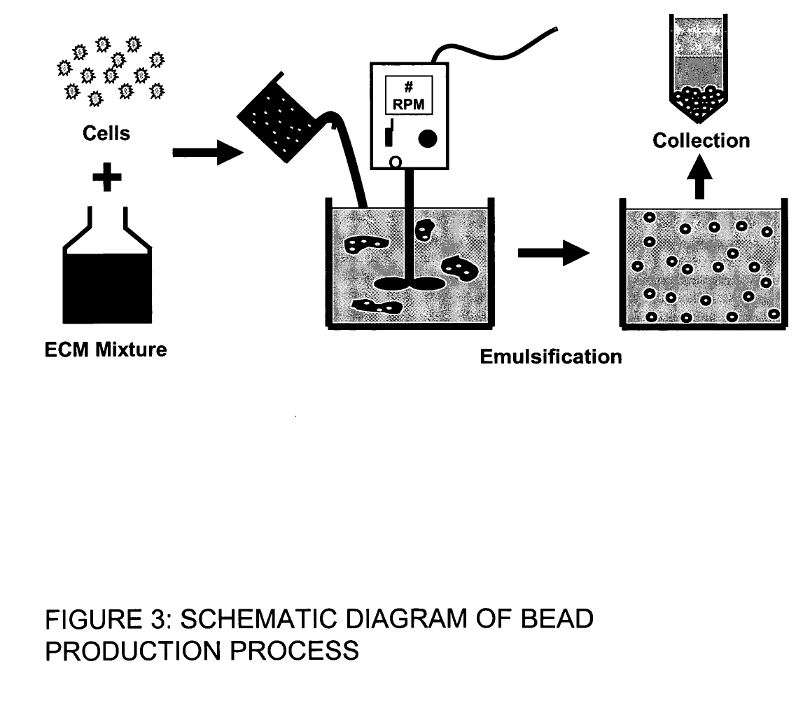

The bead production system is shown schematically in FIGS. 2 and 3. Cells were prepared for encapsulation through detachment from tissue culture flasks using trypsin-EDTA. The cells were counted and resuspended in a mixture of 5× DMEM, FBS, 0.1 M NaOH, 4.0 mg/mL collagen Type I, and 2.0% agarose, according to the formulations shown in Table I.

TABLE 1
Example formulations for the production of collagen-agarose
beads of varying collagen and agarose concentrations.

| | Col I | Agarose | NaOH | DMEM | FBS |
|---|---|---|---|---|---|
| % Col | (μL) | (μL) | (μL) | (μL) | (μL) |
| 0 | 0 | 1500 | 0 | 1200 | 300 |
| 5 | 300 | 1200 | 60 | 1140 | 300 |
| 12 | 600 | 900 | 120 | 1080 | 300 |
| 25 | 900 | 600 | 180 | 1020 | 300 |
| 40 | 1125 | 375 | 225 | 975 | 300 |
| 100 | 1500 | 0 | 300 | 900 | 300 |

During the preparation of the materials, the cells, medium and FBS were kept at 37 C. The collagen was refrigerated at 4 C until addition while agarose was heated to melt and added to the mixture at 60 C. The polymerization of collagen is both temperature and pH dependent and therefore requires the addition of NaOH (kept at 37 C) in ratio with the collagen. The collagen solution was added immediately after the addition of NaOH to neutralize the solution and prevent cellular shock. Agarose was kept heated to prevent the polysaccharide from polymerizing prior to injection into the warm PDMS emulsification phase. All components were combined at the proportions prescribed in Table I in preparation for being added to the emulsification vessel.

In this embodiment of the present invention, a cell concentration of 1.0×10⁶ cells per mL/ECM was used. The emulsification vessel consisted of a bath of PDMS fluid kept at 37 C. A stirrer motor fitted with a double bladed impeller was used to stir the bath and create a complex flow pattern that encouraged break up of liquid droplets. Stirrer speed and the distance between the impeller blades could be controlled by the operator. A secondary bath containing ice water could be fitted around the PDMS bath, in order to achieve rapid cooling and gelation of the emulsified droplets into beads. The suspension of living cells in ECM solution was rapidly injected into the warm, continuously stirred PDMS phase. Emulsification was facilitated by turning the stirrer motor off for brief periods (5-10 s) and then reinitiating stirring to break up larger cell/ECM droplets. Total emulsification time was 6 min, at which time an ice water bath was placed around the emulsification vessel and the system was cooled for 30 min with continuous stirring. This allowed for gelation of the spherical bead phase, while preventing re-agglomeration of the cell/ECM mixture. All material from the emulsification vessel was then transferred to centrifuge tubes along with an equal volume of sterile PBS, and this mixture was spun down at 1,000 rpm for 5 min. The formed cell/ECM beads preferentially partitioned into the aqueous (PBS) phase, allowing the PDMS to be aspirated away. Two similar wash steps with PBS followed in order to remove any remaining PDMS. The washed beads were transferred to culture dishes and kept in culture medium for up to 8 days prior to analysis.

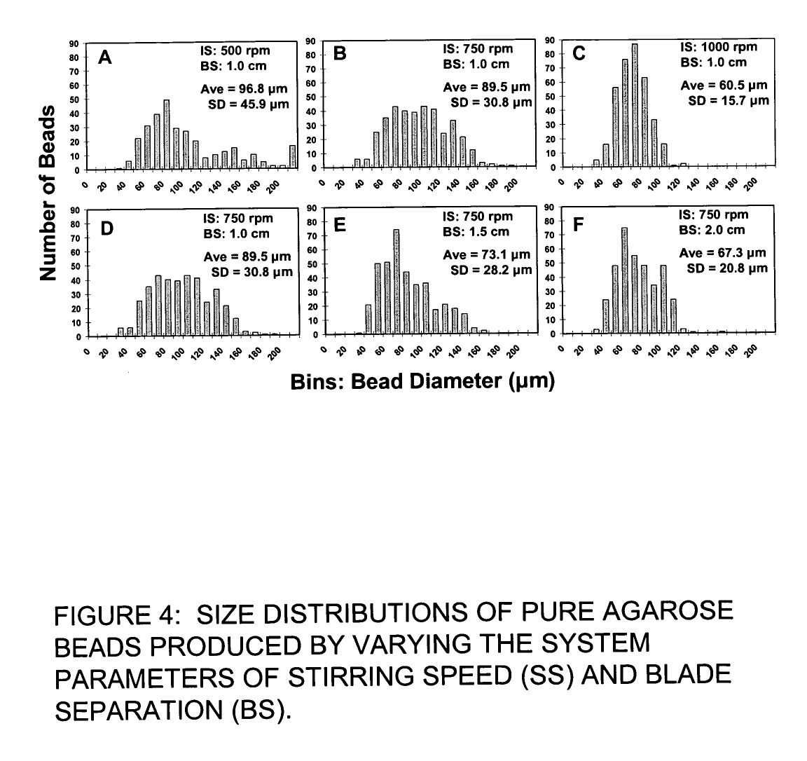

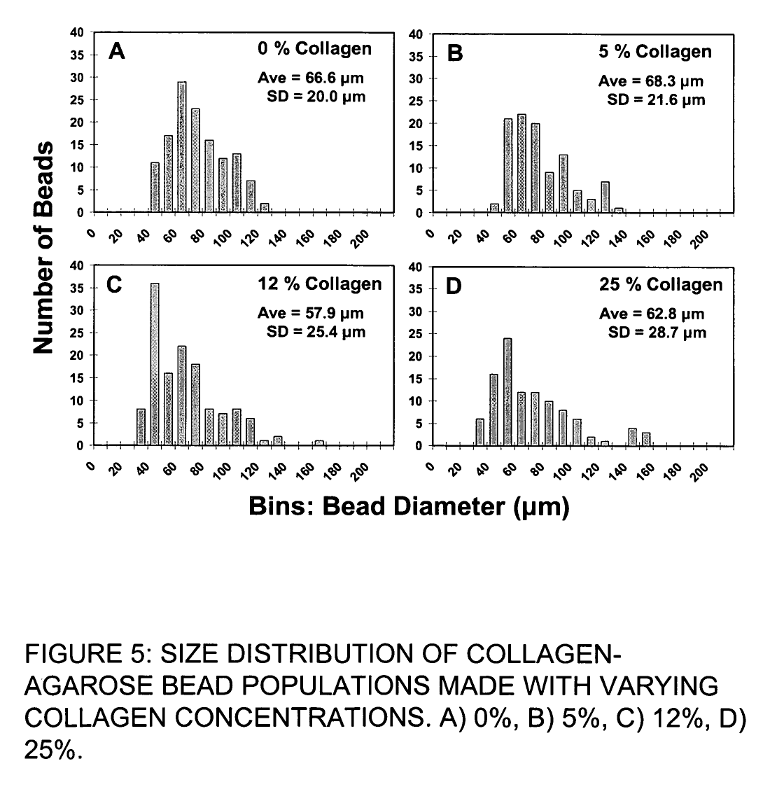

Bead size, size distribution, and cell morphology were observed by light microscopy and digital images of bead populations. FIG. 4 contains representative histograms of bead sizes, showing the effect of varying impeller speed (panels A-C) and distance between impeller blades (panels D-F) on the size and size distribution of agarose beads. All beads produced were uniformly spherical, and it can be seen that the size range was generally 20-200 microns in diameter. Increasing the impeller speed produced smaller beads with a narrower size distribution. Similarly, increasing the impeller separation yielded a narrower size distribution. The bead size histograms in FIG. 5 show the effect of increasing the proportion of collagen in beads, while keeping encapsulation parameters constant. Bead size was not significantly affected by collagen concentration, within the experimental variability of the bead production process. Further experiments were performed using collagen concentrations of 0, 12, 25, and 40%, with the balance of the matrix consisting of agarose.

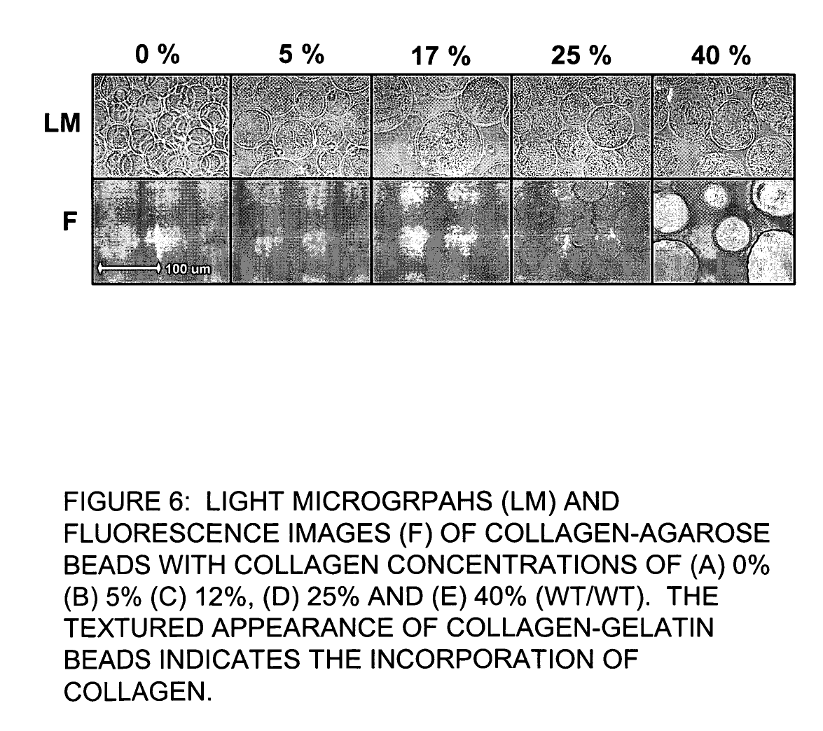

Light microscopy and fluorescent staining for Type I collagen was used to visualize and confirm the collagen content of beads as shown in FIG. 6. The beads were centrifuged and washed with PBS to remove culture medium and small samples of each bead preparation were fluorescently tagged with antibodies to collagen Type I. This examination confirmed that the collagen matrix was uniformly distributed throughout the beads. The upper panels show light micrographs of collagen-agarose beads, and the textured appearance caused by the presence of fibrillar collagen is seen to increase with increasing collagen concentration. The lower panels show fluorescently stained Type I collagen, and the increasing brightness shows that the collagen is distributed throughout the beads.

The viability of encapsulated hMSC was assessed at day 0 and day 8 using a calcein/ethidium homodimer vital stain. The viability range was estimated by examining images from several samples of each bead preparation (images are not shown because viability information is indicated by color, and these data are therefore presented as ranges in the text). Viability at day 0 was generally in the range of 75-90%, and was not markedly changed at the day 8 time point. This suggests that cell viability was not compromised by the encapsulation process and that viability remained essentially constant for at least a week.

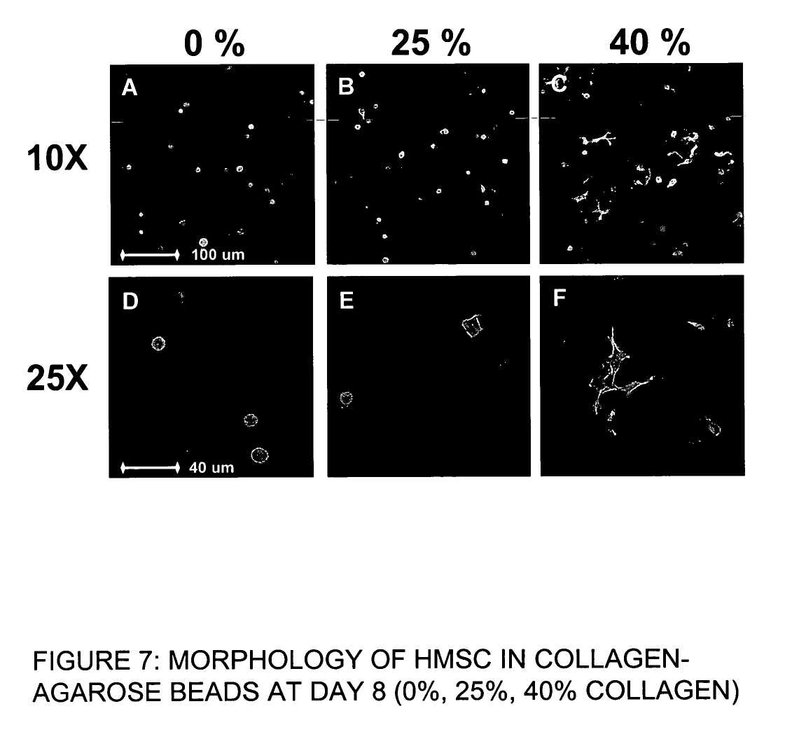

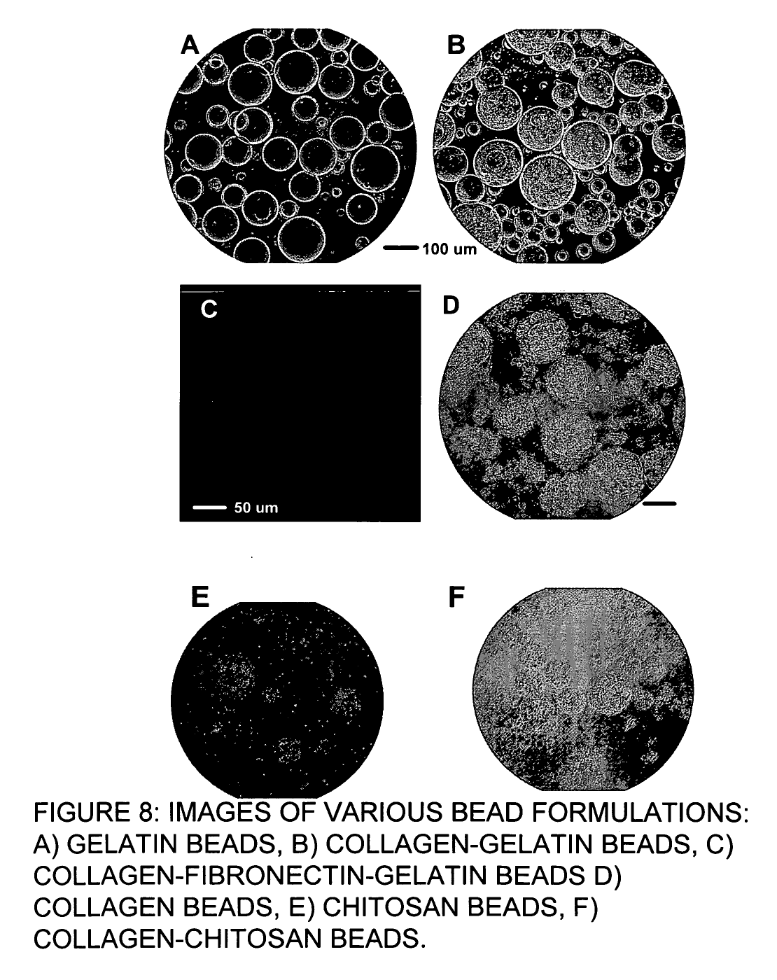

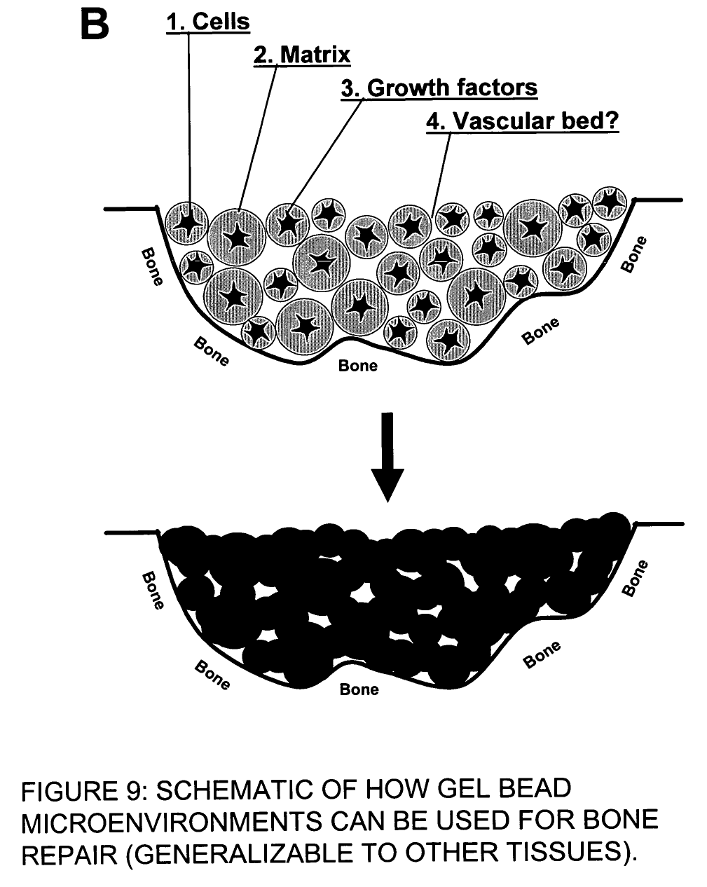

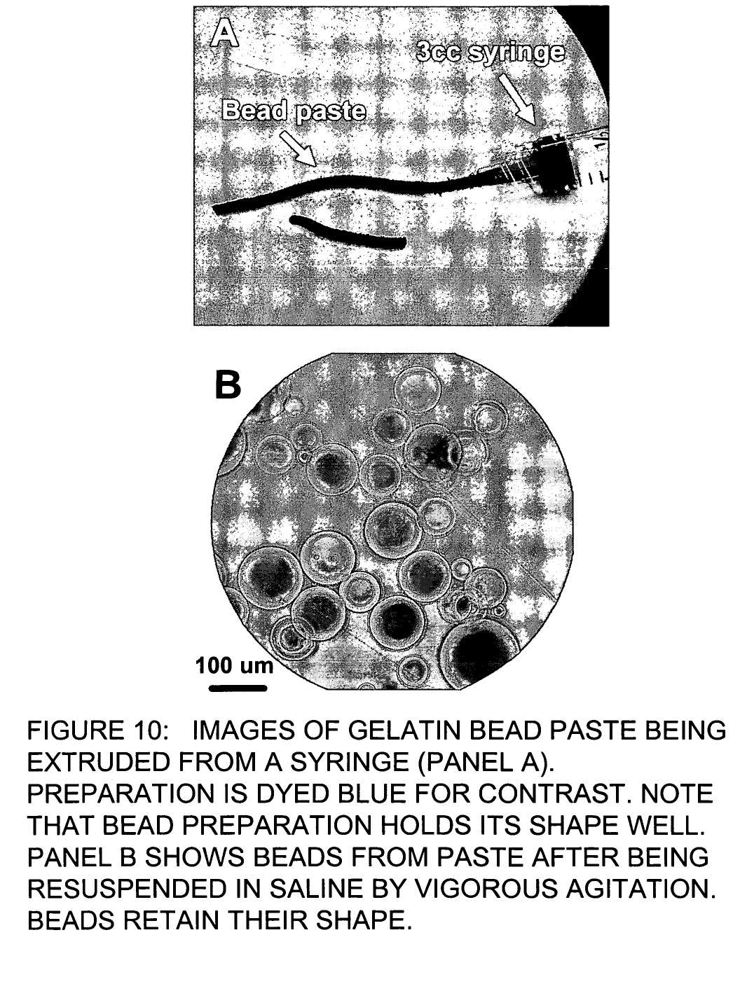

The morphology of hMSC in beads at day 8 in culture was assessed by staining the nucleus and actin cytoskeleton, as shown in FIG. 7. Immediately after encapsulation (day 0), cells in all bead types were spherical, with the same morphology they exhibited after being trypsinized and put into suspension. At day 8 in culture, cells in pure agarose beads (panels A and D) remained rounded up, with essentially no evidence of spreading or pseudopod extension. In beads containing 25% collagen (panels B and E), cells exhibited signs of spreading within the bead, but generally had a compact shape with only short cell extensions. By contrast, at day 8 in culture hMSC in 40% collagen beads (panels C and F) exhibited a markedly spread morphology with a stellate appearance and long cell protrusions.

In this example we established a reproducible procedure for creating defined 3D microenvironments for the culture of hMSC. Varying emulsification parameters such as impeller speed and blade separation provided control over the average size and size distribution of the bead populations. Bead diameters were generally in the range of 20-200 microns, and the average diameter could easily be varied from 60 to 100 microns. Beads incorporating up to 40% by weight collagen Type I could be reliably produced and harvested. Cell viability in all beads was good (75-90%) regardless of collagen content and cells cultured in beads over 8 days remained viable. As collagen content increased, hMSC exhibited increasingly spread morphologies over time in culture. Cell shape is well known to affect cell function (Chen et al., 1998; Thomas et al., 2002), and recently it has been shown that differentiation of hMSC is regulated by cell shape (McBeath et al., 2004). Control over cell shape can therefore be an important tool in regulating stem cell differentiation.

### EXAMPLE 2

### Gelatin Beads

Using a process analogous to that described in Example 1, gelatin beads can be made. In this case, a warm solution of gelatin and cells is emulsified and the emulsion is cooled to form gel beads with embedded cells. Because gelatin will re-melt when warmed to body temperature, gelatin beads can be stabilized using cross-linking to prevent remelting. Genipin or other agents that crosslink proteins (including transglutaminases and ribosylation) can be used for this purpose. FIG. 8(A) shows gelatin beads that have been stabilized with genipin cross-linking.

### EXAMPLE 3

### Collagen-Gelatin Beads

Using a process analogous to that described in Example 1, collagen-gelatin beads can be made. In this case, a warm solution of collagen, gelatin and cells is emulsified and the emulsion is cooled to form gel beads with embedded cells. Because gelatin will re-melt when warmed to body temperature, collagen-gelatin beads can be stabilized using cross-linking to prevent remelting. Genipin or other agents that crosslink proteins (including transglutaminases, ribosylation) can be used for this purpose. FIG. 8(B) shows collagen-gelatin beads that have been stabilized with genipin cross-linking. Alternately, the gelatin can be allowed to remelt after bead collection, thereby producing pure collagen beads (see Example 5, below).

### EXAMPLE 4

### Collagen-Fibronectin-Gelatin Beads

Using a process analogous to that described in Example 3, collagen-fibronectin-gelatin beads can be made. In this case, a warm solution of collagen, fibronectin, gelatin and cells is emulsified and the emulsion is cooled to form gel beads with embedded cells. Because gelatin will re-melt when warmed to body temperature, collagen-gelatin beads can be stabilized using cross-linking to prevent remelting. Genipin or other agents that crosslink proteins (including transglutaminases, ribosylation) can be used for this purpose. FIG. 8(C) shows collagen-fibronectin-gelatin beads that have been stabilized with genipin cross-linking. Alternately, the gelatin can be allowed to remelt after bead collection, thereby producing collagen-fibronectin beads (see Example 5, below).

### EXAMPLE 5

### Collagen Beads

Pure collagen beads can be made by producing and collecting collagen-gelatin beads (as in Example 3) and bypassing the gelatin stabilization (cross linking) step. When such beads are warmed above the melting point of gelatin (generally 30-40 C), the gelatin will remelt and will leave pure collagen beads. FIG. 8(D) shows collagen beads prepared by the post-gelation removal of gelatin. This is a generalizable method of using gelatin as an aid in bead collection (recovery from the hydrophobic phase), but then removing the gelatin phase to create a more pure formulation.

### EXAMPLE 6

### Chitosan Beads

Using a process analogous to that described in Example 1, chitosan beads can be made. In this case, a cold solution of chitosan in dilute acid and cells is emulsified and the emulsion is cooled to form gel beads with embedded cells. FIG. 8(E) shows chitosan beads that have been prepared in this manner.

### EXAMPLE 7

### Collagen-Chitosan Beads

Using a process analogous to that described in Example 6, collagen-chitosan beads can be made. In this case, a cold solution of collagen and chitosan in dilute acid and cells is emulsified and the emulsion is cooled to form gel beads with embedded cells. FIG. 8(F) shows collagen-chitosan beads that have been prepared in this manner.

### EXAMPLE 8

### Collagen-Chitosan-Gelatin Beads

Using a process analogous to that described in Example 6, collagen-chitosan-gelatin beads can be made. In this case, a solution of collagen, chitosan, gelatin and cells is emulsified and the emulsion is cooled to form gel beads with embedded cells. In this example, gelatin is present as a filler agent that facilitates bead collection, and the gelatin component can be retained by crosslinking or removed by temperature elevation to produce collagen-chitosan beads.

### OTHER EXAMPLES

The general methods described in Examples 1-7 are further generalizable to produce a wide range of microenvironment formulations. The proteins that can be incorporated into the microenvironment include, but are not limited to, collagen (including Types I, II, III and IV), fibrinogen, fibrin, fibronectin, gelatin, laminin, and vitronectin. In addition, the gel microenvironments can contain proteoglycan components (including hyaluronan and heparan sulfate), as well as polysaccharide components (including agar, agarose, alginate, chitosan and various combinations thereof). Each of these components can be solubilized and then gelled under conditions that are not toxic to cells, and therefore they can be used to create microenvironments with embedded living cells.

### EMBODIMENT 1

### The Example of Bone Healing

One embodiment of the present invention is the creation of 3D microenvironments that induce the osteoblastic differentiation of stem cells. Improvements in bone grafting materials offer the possibility of achieving quicker, more robust bone healing even in challenging applications such as avascular necrosis, spinal fusions and implant fixation. The ideal material for this purpose would include osteogenic cells, an osteoconductive matrix, osteoinductive growth factors, and would provide a bed for the rapid ingrowth of a vascular supply. The use of autogenous stem cells combined with fully biological matrix materials and potent, specific growth factors has the potential to produce such a material. The harvested stem cells may be embedded into protein microbeads in order to carefully define their 3D microenvironment in terms of extracellular matrix composition and growth factor availability. This will promote consistent differentiation towards the osteoblastic phenotype, and will allow cells to be injected as a concentrated slurry while still in a 3D ECM, directly to the site of bone injury.

Directed differentiation of stem cells (including embryonic, cord blood and adult mesenchymal stem cells) in defined cellular microenvironments offers several important benefits over current approaches to bone tissue engineering. The use of naturally occurring ECM proteins allows stem cells to attach to the matrix via specific integrin receptors that are transducers of both biochemical and mechanical signals. The composition of the matrix therefore can be tailored to control these signals and promote osteogenic differentiation. Incorporation of specific growth factors into the matrix immediately surrounding the cells will allow efficient, highly local delivery to cells and will further enhance osteogenic differentiation, and the response of hMSCs to these factors will be potentiated by contact with ECM proteins. The spherical shape of the beads allows them to be concentrated into a packed bed with void spaces between individual beads. When implanted into tissue, these spaces provide conduits for vascular ingrowth. Finally, the hydrogel bead format of the protein microenvironments facilitates handling and delivery of the cells and matrix as an injectable paste for the conformal filling of bone defects. FIG. 8 shows schematically how a concentrated paste of hydrogel beads might be injected into a site where bone formation is desired. Bead fusion and bone formation would occur over time, creating new bone to fill the void.

The applicants' methods for bone repair are similar in principle to others that seek to provide defined cues to promote bone formation by cells. However, the key differences are (i) the use of a full complement of ECM proteins (e.g. collagen, vitronectin, etc.); and (ii) these proteins will fully surround cells. Synthetic matrices with immobilized peptides for cell attachment, such as those developed by other groups (e.g., Hern 1998; Rowley 2002; Nuttelman 2005), have the advantage that one can control the presentation of the ligand. In most cases, the short peptide sequence RGD (arginine-glycine-aspartic acid) is used as the preferred ligand for cell attachment. However, RGD binding alone is unlikely to be sufficient to induce consistent osteogenesis. A full complement of proteins will generate an enhanced response, relative to small peptides. Indeed applicants' recent preliminary data shows that hMSCs react differently depending on which ECM protein they are exposed to. For example, hMSCs cultured on fibronectin (which contains RGD sites) do not undergo osteogenic differentiation, whereas those cultured on collagen I or vitronectin do.

The applicants' method of using directed differentiation of stem cells for bone repair addresses the four key features that are critical to effective bone repair: 1] the presence of living osteogenic cells to populate the new bone (using autogenous hMSC), 2] an appropriate extracellular matrix to provide an osteoconductive scaffold (using the natural ECM proteins collagen I and vitronectin), 3] osteoinductive and angiogenic growth factors to provide signals to the resident cells (using efficient local delivery of e.g. BMP and VEGF), and 4] an adequate blood supply to support cell growth and function (by providing void spaces between beads for vascular ingrowth).

### EMBODIMENT 2

### The Example of Heart Muscle Repair

Another embodiment of the present invention is the application of this technology in the creation of 3D microenvironments that induce differentiation of stem cells into functional cardiomyocytes. Mature cardiomyocytes do not divide, and therefore cannot regenerate areas of infarcted heart. Direct cell transplantation to injured areas of the heart has been proposed as a solution to this problem, but the issue of cell source is critical. Harvesting of mature autologous or allogeneic cardiac myocytes is not practical. Transplantation of skeletal myoblasts into the heart has been proposed and attempted, but serious problems related to the generation of arrhythmias have occurred. Stem cells have the potential to circumvent these issues and are therefore one of the most promising cell sources for cardiac regeneration therapy.

Differentiation of hMSCs into cardiomyocytes has been demonstrated in culture using the demethylating agent 5-azacytidine. This biochemical additive is used as a cancer therapeutic, but its cytotoxic effects make its use in heart failure patients questionable. There is clearly an important need to better define 3D systems that will lead to consistent, controlled differentiation of stem cells into heart muscle. Our invention can produce controlled cellular microenvironments consisting of physiological matrix proteins and biochemical factors that surround stem cells, thereby promoting the controlled differentiation of stem cells into cardiomyocytes in 3D culture. Furthermore, our gel microbeads can be used to deliver cells directly to the infarcted heart as a concentrated paste.

The ideal cell delivery system for myocardial repair would include functional cardiomyocytes in an appropriate extracellular matrix and would allow the rapid ingrowth of a vascular supply. The ability to harvest the required cells and deliver them via minimally invasive procedures also would be attractive features of such a cardiac repair system. The use of autogenous hMSC combined with fully biological matrix materials has the potential to produce such a therapy. The invention allows harvested hMSC to be embedded in protein microbeads that create a defined 3D extracellular microenvironment in terms of both extracellular matrix composition and biochemical stimulation. This system is designed to promote consistent differentiation towards the cardiomyocyte phenotype, and will further allow cells to be injected as a concentrated slurry directly to the site of heart muscle injury while still in a 3D ECM. The hydrogel bead format of the protein microenvironments facilitates handling and delivery of the cells and matrix as an injectable slurry that may provide better engraftment into damaged tissues, relative to cells implanted alone, because of the presence of appropriate matrix and biochemical components.

### OTHER EMBODIMENTS

The methods described in the embodiments above may be extended to directing the differentiation of stem cells to a variety of tissue types including bone, cartilage, fat, skeletal muscle, smooth muscle, endothelial tissue, epithelial tissue, heart muscle and nerve. In each case, the composition of the microenvironment (insoluble matrix and biochemical factors) can be tailored to promote the desired lineage.

Summary

The bead production method developed by the inventors can create 3D microenvironments containing physiologically relevant matrices that can be used to direct cell function and differentiation. The bead format provides a defined 3D matrix surrounding the cell, and has the additional advantage that relatively small amounts of matrix polymer are needed. Incorporation of such bioactive proteins in a bead matrix may allow greater control of matrix-mediated cell differentiation. In addition, growth factors and other biochemicals could be added directly into the bead matrix, providing highly local stimulation of the entrapped cells, and further enhancing control over cell differentiation. The bead format is beneficial because it allows clear definition of the local extracellular environment. The bead format also allows collection of the embedded cells while still in their preferred microenvironment, and cells in beads can be delivered as a concentrated paste for therapeutic application.

The key innovations of the present invention that differentiate it from the prior art are: (i) the use of a full complement of naturally-derived ECM proteins to control cell function, (ii) the embedding of living cells directly inside hydrogel beads made of these proteins, (iii) the use of these microenvironments to direct the differentiation of embedded stem cells, and (iv) the use of beads as a delivery system for embedded cells directly to the site of desired tissue regeneration.

The claimed bead microenvironment technology has other potential applications, including a variety of areas where control of cell function is important, such as cell-based diagnostics, therapeutic protein production via biotechnology and suspension culture of attachment-depended cells. Incorporation of other matrix materials (including proteins, proteoglycans, and polysaccharides) is within the scope of the instant invention. These materials may be selected from the group consisting of collagen (including Types I, II, III, IV, etc.), fibrinogen, fibrin, fibronectin, gelatin, laminin, and vitronectin, hyaluronan, heparan sulfate, agar, agarose, alginate, chitosan and various combinations thereof.

It is understood that the invention is not limited to the disclosed compositions, methods of preparation, treatment applications and embodiments shown, including any embodiments that may be apparent to one of ordinary skill in the art. Although the foregoing invention has been described in some detail by way of illustration for purposes of clarity of understanding, it will be readily apparent to those of ordinary skill in the art in light of the teachings of this invention that certain variations and modifications may be made thereto without departing from the spirit or scope of the disclosure herein, including the specific embodiments. In other words, numerous other variations of the present invention will be appreciated by those skilled in the art in view of the disclosure herein.

We claim:

1. A method of encapsulating stem cells within three-dimensional polymer microenvironments, wherein said polymer is a naturally derived polymer obtained from living organisms, wherein said method comprises the following steps:
(i) mixing a polymer solution with stem cells in a buffer to incorporate and disperse the stem cells therein to form a cell-liquid suspension,
(ii) emulsifying said cell-liquid suspension in a hydrophobic fluid phase to form an emulsified liquid,
(iii) initiating polymerization of said emulsified liquid to form three-dimensional polymer microenvironments, and
(iv) collecting the three-dimensional polymer microenvironments from the hydrophobic phase.

2. The method of claim 1, further comprising culturing the three-dimensional polymer microenvironments to promote differentiation of the stem cells into cells of a specific tissue type, wherein said tissue is selected from the group consisting of bone, cartilage, fat, skeletal muscle, smooth muscle, endothelial, epithelial, heart, muscle and nerve.

3. The method according to claim 1, wherein said hydrophobic fluid phase is selected from the group consisting of silicone oil, mineral oil, vegetable oil, room-temperature ionic liquids and combinations thereof.

4. The method according to any one of claims 1 or 2, wherein said polymer is selected from the group consisting of collagen, elastin, fibrinogen, fibrin, fibronectin, gelatin, laminin, vitronectin, hyaluronan, heparan sulfate, agar, agarose, alginate, chitosan and combinations thereof.

5. The method according to any one of claims 1 or 2, wherein said polymer is selected from the group consisting of collagen-gelatin, collagen-agarose, collagen-chitosan, collagen-chitosan-agarose, collagen-chitosan-gelatin, collagen-vitronectin-agarose, collagen-vitronectin-gelatin, collagen-vitronectin-chitosan, collagen-fibronectin-agarose, collagen- fibronectin-gelatin collagen- fibronectin-chitosan, collagen-laminin-agarose, collagen-laminin-gelatin, collagen-laminin-chitosan.

6. The method of claim 1, which further comprises incorporating an additional agent selected from the group consisting of excipients, growth factors, vitamins, minerals, ions, gases, crosslinking agents, active agents, carriers and combinations thereof.

7. The method of claim 6, wherein the gases are selected from the group consisting of oxygen, nitrogen and mixtures thereof.

8. The method of claim 1, wherein said three-dimensional polymer microenvironment is a crosslinked hydrogel system.

9. The method of claim 1, wherein said three-dimensional polymer microenvironment is crosslinked using genipin, transglutaminase, ribosylation and combinations thereof.

10. The method according to claim 1, wherein said method further includes adding a polymeric filler material prior to step (iv) to aid in harvesting of the three-dimensional polymer microenvironments, wherein said polymeric filler material is removed following collection of the three-dimensional polymer microenvironments.

11. The method according to claim 10, wherein said polymeric filler material comprises gelatin.

12. The method of claim 1, wherein said stem cells are obtained from bone marrow or cord.

13. The method of claim 1, wherein said stem cells are adult stem cells.

14. The method of claim 1, wherein said stem cells are human mesenchymal stem cells (hMSCs).

15. The method of claim 1, wherein said stem cells are embryonic stem cells.

16. The method according to any one of claims 1 or 2, wherein said polymer microenvironments are beads having a size range of 20-1000 microns in diameter.

17. The method according to any one of claims 1 or 2, wherein said polymer microenvironments are beads having a size range of 20-200 microns in diameter.

18. A method of producing a population of differentiated viable stem cells, wherein said method comprises directed differentiation of stem cells by the following steps:
(i) mixing a polymer solution derived from living organisms with stem cells in a buffer to incorporate and disperse the stem cells therein to form a cell-liquid suspension,
(ii) emulsifying said cell-liquid suspension in a hydrophobic fluid phase to form an emulsified liquid,
(iii) initiating polymerization of said emulsified liquid to form three-dimensional polymer microenvironments,
(iv) collecting the three-dimensional polymer microenvironments via centrifugation, and
(v) culturing said three-dimensional polymer microenvironments to promote the directed differentiation of the embedded stem cells.

19. The method according to claim 18, wherein said hydrophobic fluid phase is selected from the group consisting of silicone oil, mineral oil, vegetable oil, room temperature ionic liquids and combinations thereof.

20. The method according to claim 18, wherein said polymer is selected from the group consisting of collagen, elastin, fibrinogen, fibrin, fibronectin, gelatin, laminin, vitronectin, hyaluronan, heparan sulfate, agar, agarose, alginate, chitosan and combinations thereof.

21. The method according to claim 18, wherein said polymer is selected from the group consisting of collagen-gelatin, collagen-agarose, collagen-chitosan, collagen-chitosan-agarose, collagen-chitosan-gelatin, collagen-vitronectin-agarose, collagen-vitronectin-gelatin, collagen-vitronectin-chitosan, collagen-fibronectin-agarose, collagen-fibronectin-gelatin collagen-fibronectin-chitosan, collagen-laminin-agarose, collagen-laminin-gelatin, collagen-laminin-chitosan.

22. The method of claim 18, wherein said three-dimensional polymer microenvironment is crosslinked using genipin, transglutaminase, ribosylation and combinations thereof.

23. The method according to claim 18, wherein said three-dimensional polymer microenvironment is a hydrogel bead.

24. The method of claim 18, wherein said stem cells are obtained from bone marrow or cord/placental blood.

25. The method of claim 18, wherein said stem cells are adult stem cells.

26. The method of claim 18, wherein said stem cells are human mesenchymal stem cells (hMSCs).

27. The method according to claim 18, wherein said polymer microenvironments are beads having a size range of 20-1000 microns in diameter.

28. The method according to claim 18 wherein said polymer microenvironments are beads having a size range of 20-200 microns in diameter.

29. The method of claim 18, which further comprises incorporating an additional agent selected from the group consisting of excipients, growth factors, vitamins, minerals, ions, gases, cross-linking agents, active agents, carriers and combinations thereof.

30. The method of claim 29, wherein the gases are selected from the group consisting of oxygen, nitrogen and mixtures thereof.

31. A method of encapsulating and delivering stem cells within hydrogel polymer microenvironments, wherein said method comprises the following sequential steps:
(i) mixing a polymer derived from living organisms with stem cells in a buffer to incorporate and disperse the stem cells therein to form a cell-liquid suspension,
(ii) emulsifying said cell-liquid suspension in a hydrophobic fluid phase to form an emulsified liquid,
(iii) initiating polymerization of said emulsified liquid to form polymer microenvironments,
(iv) collecting the polymer microenvironments from the hydrophobic phase,
(v) forming a concentrated paste of said polymer microenvironments, and
(vi) loading the paste into a syringe and extruding it for cosmetic or therapeutic application to a subject in need thereof.

32. The method according to claim 31, wherein said hydrophobic fluid phase is selected from the group consisting of silicone oil, mineral oil, vegetable oil, room temperature ionic liquids and combinations thereof.

33. The method according to claim 31, wherein said polymer is selected from the group consisting of collagen, elastin, fibrinogen, fibrin, fibronectin, gelatin, laminin, vitronectin, hyaluronan, heparan sulfate, agar, agarose, alginate, chitosan and combinations thereof.

34. The method according to claim 31, wherein said polymer is selected from the group consisting of collagen-gelatin, collagen-agarose, collagen-chitosan, collagen-chitosan-agarose, collagen-chitosan-gelatin, collagen-vitronectin-agarose, collagen-vitronectin-gelatin, collagen-vitronectin-chitosan, collagen-fibronectin-agarose, collagen-fibronectin-gelatin collagen-fibronectin-chitosan, collagen-laminin-agarose, collagen-laminin-gelatin, collagen-laminin-chitosan.

35. The method of claim 31, which further comprises incorporating an additional agent selected from the group consisting of excipients, growth factors, vitamins, minerals, ions, gases, cross-linking agents, active agents, carriers and combinations thereof.

36. The method of claim 35, wherein the gases are selected from the group consisting of oxygen, nitrogen and mixtures thereof.

37. The method of claim 31, wherein said stem cells are obtained from bone marrow or cord blood.

38. The method according to claim 31, wherein said method produces beads having a size range of 20-1000 microns in diameter.

39. The method according to claim 31, wherein said method produces beads having a size range of 20-200 microns in diameter.

40. The method according to claim 31, wherein said therapeutic application is tissue repair or regeneration.

41. The method according to claim 40, wherein said tissue repair or regeneration is selected from bone repair, nerve repair, cardiac muscle repair, skin repair and cartilage repair.

42. The method of claim 31, wherein the application of said paste is by injection or implantation directly to the application site.

43. The method of claim 31, wherein the application of said paste is directly to the site of bone injury to promote differentiation towards the osteoblastic phenotype.

44. The method of claim 31, wherein said therapeutic application causes directed differentiation of stem cells into cells selected from the group consisting of osteoblasts, chondroblasts, adipocytes, fibroblasts, endothelial cells, neurons, smooth muscle cells, skeletal myoblasts and cardiac myocytes.

45. The method of claim 44, wherein differentiation towards the osteoblastic phenotype results in therapies involving avascular necrosis, spinal fusion or implant fixation.
